(19) United States
(12) Patent Application Publication
(10) Pub. No.: US 2003/0120225 A1
(43) Pub. Date: Jun. 26, 2003

(54) Therapeutic agent delivery labial pad

(75) Inventors: Dennis Stein Everhart, Alpharetta, GA (US); Jack Nelson Lindon, Alpharetta, GA (US); Kimberly Marie Geiser, Appleton, WI (US); David William Koenig, Menasha, WI (US); Bernard Joseph Minerath, Oshkosh, WI (US); Barbara Jo Dvoracek, Appleton, WI (US); David John Tyrrell, Appleton, WI (US); Duane Gerard Krzysik, Appleton, WI (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/027,267

(22) Filed: Dec. 21, 2001

(51) Int. Cl. A61M 31/00; A61F 13/15; A61F 13/20

(52) U.S. Cl. 604/285; 604/367; 604/385.17

(57) ABSTRACT

An absorbent device configured for partial disposition within the vestibule of a wearer, and adapted to deliver a therapeutic agent, the device including a fluid-absorbent body having an application region for projection within the vestibule; and a formulation including a therapeutic agent positioned substantially within the application region. Also, a method for producing an absorbent device configured for partial disposition within the vestibule of a wearer, and adapted to deliver a therapeutic agent, the method including manufacturing an absorbent device having a fluid-absorbent body having an application region for projection within the vestibule; and locating a formulation including the therapeutic agent substantially within the application region.

### BACKGROUND

The present invention relates generally to absorbent articles. More particularly, the present invention relates to absorbent articles such as labial pads configured for disposition within the vaginal vestibule of a female wearer and configured to deliver a therapeutic agent.

Many disease states and physiological conditions can occur in a woman, including symptoms associated with premenstrual syndrome, menstruation, and menopause. These symptoms may include dysmenorrhea (menstrual cramping), irritability, water retention, moodiness, depression, anxiety, skin changes, headaches, breast tenderness, tension, weight gain, cravings, fatigue, and hot flashes. Symptoms of conditions can include itching and other associated sensory maladies.

Many of these symptoms are due to changes in hormonal levels throughout the menstrual cycle. Menstrual cramping is associated with increased levels of prostaglandin F2α, prostaglandin E2, and in some cases leukotrienes in the endometrium and menstrual fluid. These eicosinoids lead to restricted blood flow to the uterus and increased uterine contractions, causing pain.

One symptom is dysmenorrhea, which is the occurrence of painful uterine cramps during menstruation that affects a large number of post-pubescent women. The pain of dysmenorrhea originates in the uterus. Various analgesics can be effective in limiting the pain from dysmenorrhea; some have used orally-delivered analgesics, while others have searched for alternative analgesic delivery methods. Attempts have been made to deliver analgesics in the vicinity of the cervix, the uterus, and the vaginal mucosa using various vaginally-inserted and intrauterine devices and methods. A similar situation exists with many other disease states and physiological conditions.

Disposable absorbent devices for the absorption of human exudates are widely used. These disposable absorbent devices typically have a mass of absorbent formed into a desired shape, which is typically dictated by the intended consumer use.

A broad manner and wide variety of absorbent articles configured for the absorption of bodily exudates such as menstrual fluid are well known. With respect to feminine hygiene, the art has offered two basic types of feminine hygiene protection: sanitary napkins, developed for external wear about the pudendal region, and tampons, developed for residence within the vaginal cavity and interruption of menstrual flow therefrom. Hybrid feminine hygiene protection devices, attempting to merge the structural features of both within a single type of device, have also been proposed, but have not seen a meaningful measure of acceptance insofar as the effort to appropriate advantages has been overshadowed by the more demonstrable perpetuation of structural and anatomically functional disadvantages. Other less intrusive devices, known as labial or interlabial devices and characterized as having a portion which at least partially resides external of the wearer's vestibule, have also been proposed.

Because dysmenorrhea typically occurs in conjunction with menstruation, some have tried to combine an analgesic with a tampon such that the tampon can perform two functions: absorption and treatment.

Many of these prior devices have not fully satisfied the demand of consumers for even smaller devices that may be worn interlabially by female wearers. In response thereto, several manufacturers have produced labial pads that are quite small in size in comparison to the prior devices described above.

### SUMMARY

One difficulty in using orally-delivered analgesics is that oral doses of analgesics large enough to be efficacious can lead to adverse side effects, thus limiting the actual use of the analgesics. Limiting doses in an attempt to limit those side effects results in an insufficient amount of analgesic delivered to the uterus. In addition, the use of analgesics delivered by alternative means, including through the use of absorbent articles, can still cause side effects because of the inherent nature of the analgesics.

The difficulty in using an absorbent article to deliver a therapeutic agent is managing the transfer of a therapeutic agent out of or from the absorbent article, and the transfer of menstrual fluid or other body fluid exudates into the absorbent article. For example, if the therapeutic agent formulation is generally hydrophilic, the therapeutic agent formulation will tend to absorb into the absorbent article, or be carried by the menses into the absorbent article. If the therapeutic agent formulation is generally hydrophobic, the therapeutic agent formulation will tend to block the absorbency of the absorbent article, especially if the therapeutic agent is applied to the distal end of the absorbent article, which is the end closest to the source of menses. Both of these effects compromise precise dosing of the therapeutic agent to the user of the absorbent product

The present invention overcomes these problems by providing an absorbent article that delivers a therapeutic agent without affecting the absorbency of the absorbent article.

This invention describes a therapeutic agent delivery system in cooperation with an absorbent article, such that the absorbent functionality of the absorbent article is preserved in addition to providing an integral therapeutic agent delivery system. The therapeutic agent delivery system including the therapeutic agent and carrier components can be any therapeutic agent that will be absorbed into the body through the labial epithelium, for the purposes of treating dysmenorrhea or other conditions. One embodiment is for the therapeutic agent and its delivery system to be applied to the outer surface of the absorbent article and predominantly to the surfaces that are in contact with the vaginal epithelium. Other embodiments would provide a reservoir of a therapeutic agent incorporated into the absorbent article to provide for varying doses, or a means to provide release of the therapeutic agent over the duration of contact with the vaginal epithelium.

More specifically, the invention provides an absorbent device configured for partial disposition within the vestibule of a wearer, and adapted to deliver a therapeutic agent, the device including a fluid-absorbent body having an application region for projection within the vestibule; and a formulation including a therapeutic agent positioned substantially within the application region. The invention also provides a method for producing an absorbent device configured for partial disposition within the vestibule of a wearer, and adapted to deliver a therapeutic agent, the method including manufacturing an absorbent device having a fluid-absorbent body having an application region for projection within the vestibule; and locating a formulation including the therapeutic agent substantially within the application region. The invention further provides a method of delivering a therapeutic agent through the non-cornified epithelium of the labia of a wearer. The method includes disposing an absorbent device at least partially within the vestibule of the wearer, wherein the device is adapted to contact the non-cornified epithelium and deliver the therapeutic agent.

Other objects and advantages of the present invention will become more apparent to those skilled in the art in view of the following description and the accompanying drawings.

### DRAWINGS

The foregoing and other features, aspects and advantages of the present invention will become better understood with regard to the following description, appended claims and accompanying drawings where:

FIG. 1 is a simplified anatomical cross-sectional view of a human female illustrating the environment for an absorbent article of the present invention.

FIG. 1A is a simplified anatomical cross-sectional view of a human female illustrating a placement of an absorbent article disposed in the vestibule of a wearer.

FIG. 2 is a top view illustrating an embodiment of an absorbent article.

FIG. 3 is cross-sectional view of the absorbent article illustrated in FIG. 2 taken along line 3-3 thereof.

FIG. 4 is a cross-sectional view illustrating another embodiment of an absorbent article.

FIG. 5 is a top view illustrating an embodiment of an absorbent article similar to that illustrated in FIG. 2.

FIG. 6 is a cross-sectional view illustrating an embodiment of an absorbent article.

FIG. 7 is a cross-sectional view illustrating the embodiment of FIG. 6 in a folded position.

FIG. 8 illustrates an exaggerated enlarged view of an embodiment of an absorbent article folded along a desired axis of flexure and being grasped for disposition in the vestibule by the wearer's fingers.

### DETAILED DESCRIPTION OF THE PREFERRED EMBODIMENTS

The structure of the device described herein is essentially similar to the devices described in co-pending applications U.S. Patent Application Serial Nos. 60/297,002, “Labial Pad,” and 60/315,257 “Absorbent Labial Pad,” which are incorporated herein by reference. Alternate embodiments of absorbent articles are disclosed therein.

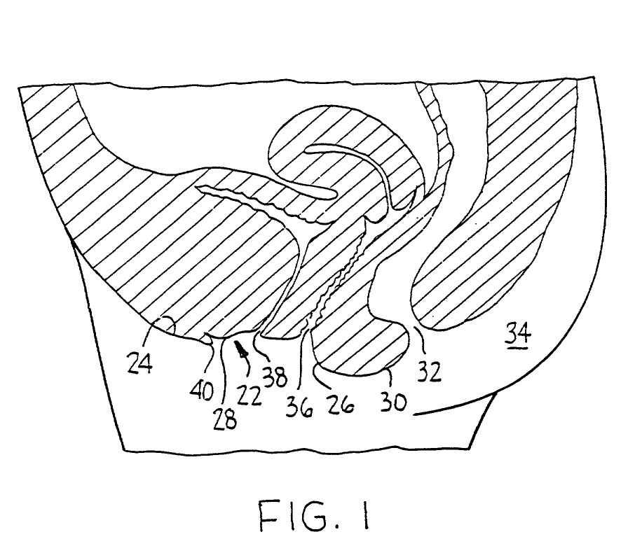

Turning to the figures of drawing, i.e., FIGS. 1 through 8, in each of which similar parts are identified with like reference characters, FIG. 1A illustrates diagrammatically an absorbent article, such as a labial pad, of the present invention, designated generally as 20, disposed within the vestibule of a wearer, designated generally as 22 (see FIG. 1). As used herein, the term “labial pad” refers to a device having a least some absorbent components, and which is specifically configured for disposition in between the labia majora, extending at least partially into the vestibule (22) of a female wearer during use. For purposes of the ensuing description, the vestibule (22) is considered to be the region defined within the labia (not specifically shown in the figures herein) beginning at about a point lying caudally from the anterior labial commissure (24), extending rearward to the posterior labial commissure (26) and bounded inwardly by the floor (28). One of skill in the art fully understands that there is a wide range of variation among women with respect to the relative size and shape of labia majora and labia minora as the same interrelatedly define the contour of the vestibule (22). For purposes of the present description, however, such differences will not specifically be addressed, it being recognized that in any event the disposition of the absorbent article (20) of the present invention into the vestibule (22) will necessitate placement between the labia majora regardless of any such consideration respecting the labia minora. Lying caudally of the vestibule (22) is the perineum (30), which leads to the anus (32) in the region of the buttocks (34). Within the vestibule (22) itself is located the principal urogenital members which, for purposes pertinent here, are constituted of the vaginal orifice (36), the urethral orifice (38), and the clitoris (40). Given the foregoing simplified review of this anatomical region, and to facilitate the present description, the vestibule (22) will be considered generally to be the region between the clitoris (40) and the posterior labial commissure (26), for convenience.

For a more comprehensive description of this portion of the human female anatomy, however, attention is invited to Anatomy of the Human Body by Henry Gray, Thirtieth American Edition (Carmine D. Clemente ed., Lea & Febiger, 1985), at 1571-1581.

As can be seen with reference to the anatomical structure illustrated in FIGS. 1 and 1A, the absorbent article (20) of the present invention is disposed at least partially within the vestibule (22) for at least partially occluding the same respecting fluid flow therefrom. In this regard, the predominant use of the absorbent article (20) is for the absorption of menstrual fluid emitted via the vaginal orifice (36); although the absorbent article of the present invention is equally well adapted to serve as a type of incontinence device for absorption of urine as occurs upon minor female incontinence.

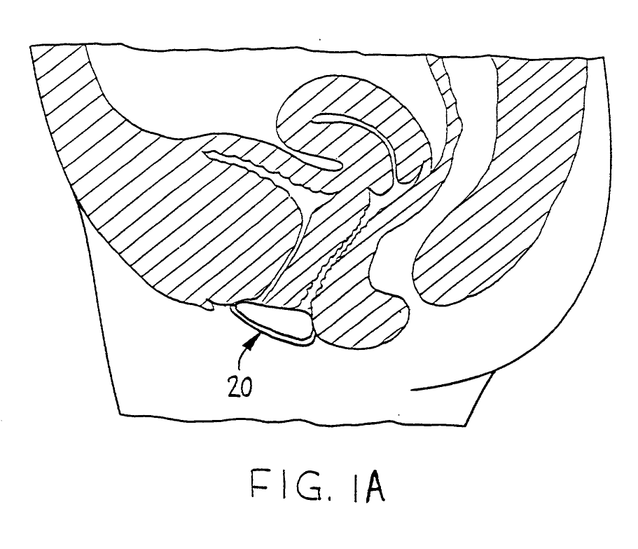

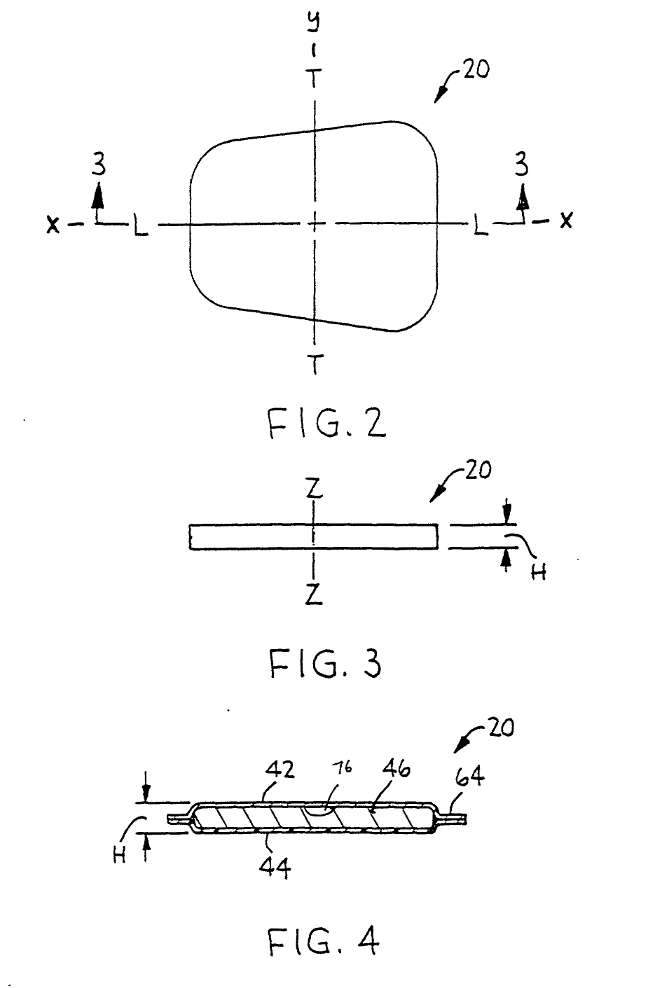

The absorbent article (20) of the present invention, as generally illustrated in FIG. 2 by way of example, has a principal longitudinal axis (L), which generally runs along the x direction. As used herein, the term “longitudinal” refers to a line, axis or direction in the plane of the absorbent article (20) that is generally aligned with (e.g., approximately parallel to) a vertical plane that bisects a standing female wearer into left and right body halves when the absorbent article is in use. The longitudinal direction is generally illustrated in FIG. 2 by the x-axis. The absorbent article (20) also has a principal transverse axis (T). The terms “transverse,” “lateral” or “y direction” as used herein generally refer to a line, axis or direction that is generally perpendicular to the longitudinal direction. The lateral direction is generally illustrated in FIG. 2 by the y-axis. The “z direction,” generally illustrated in FIG. 3, is a line, axis or direction generally parallel to the vertical plane described above. The z direction is generally illustrated in FIG. 3 by the z-axis. The term “upper” refers generally to an orientation directed toward the wearer's head, while the terms “lower” or “downwardly” refer generally to an orientation directed toward the wearer's feet. For purposes of discussion herein, each layer of the absorbent article (20), e.g., the fluid permeable cover (42), the liquid impermeable baffle (44) and the absorbent (46), has an upper or body-facing surface and a lower surface also described as the surface opposed to the upper or body-facing surface.

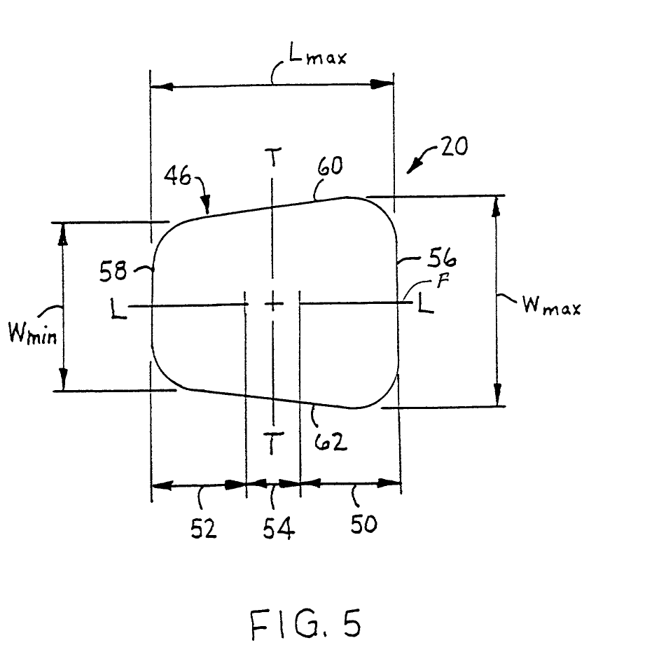

Turning now to FIG. 4, an absorbent article (20) is illustrated as comprising a fluid permeable cover (42), a liquid impermeable baffle (44) and an absorbent (46) situated between the cover and the baffle. As illustrated in FIG. 5, the absorbent (46) has a first end region (50), a second end region (52) and a central region (54) disposed between each of the end regions. The absorbent article (20) should be of a suitable size and shape that allows at least a portion of the absorbent article to be disposed within the vestibule (22) of a female wearer. In addition, the absorbent article (20) desirably at least partially occludes and intercepts the flow of menstrual fluid, urine or other bodily exudates from the wearer's vaginal orifice (36) and/or urethral orifice (38).

The absorbent (46), and thus the absorbent article (20), generally displays a geometry extending between spaced-apart first (56) and second (58) transverse end areas. The overall geometry is completed by noting that the absorbent (46), and thus the absorbent article (20), also includes spaced apart first (60) and second (62) longitudinal sides ranging between the transverse end areas (56, 58), these collectively sometimes being referred to herein as the perimetral sides (i.e., those defining the perimeter).

The geometry of the absorbent (46) is a significant factor affecting the overall size and effectiveness of the absorbent article (20). In general, the absorbent (46) has a maximum width (Wₘₐₓ), measured along a line laying generally parallel to the principal transverse axis (T) and running from one longitudinal side to the opposing longitudinal side (60, 62), and a minimum width (Wₘᵢₙ), measured along a line also laying generally parallel to the principal transverse axis (T) and running from one longitudinal side to the opposing longitudinal side (60, 62). The maximum width (Wₘₐₓ) of the absorbent (46) can be situated in the first (50) and/or second (52) end regions, while the minimum width (Wₘᵢₙ) of the absorbent (46) is situated in a region or regions other than the region or regions in which the maximum width (Wₘₐₓ) of the absorbent is situated. For example, when the maximum width (Wₘₐₓ) of the absorbent (46) is situated in the first end region (50), the minimum width (Wₘᵢₙ) of the absorbent (46) can be situated in either the second end region (52), the central region (54), or in both the second end and central regions. Alternatively, when the maximum width (Wₘₐₓ) of the absorbent (46) is situated in the second end region (52), the minimum width (Wₘᵢₙ) of the absorbent (46) can be situated in either the first end region (50), the central region (54), or in both the first end and central regions. In another alternative, when the maximum width (Wₘₐₓ) of the absorbent (46) is situated in both the first (50) and second (52) end regions, the minimum width (Wₘᵢₙ) of the absorbent (46) is situated in the central region (54). It has been found (for reasons discussed further herein) that it is not desirable to have the maximum width of the absorbent (46) situated in the central region (54). The absorbent (46) may have a width ranging between no less than about 5 mm up to no greater than about 70 mm; although the approximate width(s) of the absorbent will vary according to, inter alia, the general design and intended disposition of the absorbent article (20) within the vestibule (22) of a female wearer.

The absorbent (46) has a maximum length (Lₘₐₓ), measured along a line laying generally parallel to the principal longitudinal axis (L) and running from one transverse end area to the other transverse end area. The absorbent (46) may also have a minimum length (Lₘᵢₙ), measured along a line also laying generally parallel to the principal longitudinal axis (L) and running from one transverse end area to the other transverse end area. The absorbent (46) may have a length ranging between no less than about 40 mm up to no greater than about 100 mm; although the approximate length(s) of the absorbent will vary according to, inter alia, the general design and intended disposition of the absorbent article (20) within the vestibule (22) of a female wearer. One of skill in the art will readily appreciate that the absorbent (46), and thus the absorbent article (20), of the present invention may have minimum length (Lₘᵢₙ) equal to its maximum length (Lₘₐₓ). In such instances, as illustrated at least in FIG. 5, reference is generally made only to the maximum length (Lₘₐₓ).

The first end region (50) and the second end region (52) each minimally extend outwardly from the central region (54) toward the transverse end areas (56 and 58, respectively) of the absorbent (46). The end regions (50, 52) may occupy from a minimum of about 20% up to a maximum of about 80% of the maximum length (Lₘₐₓ) of the absorbent (46); although the approximate size of the first and second end regions will vary according to, inter alia, the general design and intended disposition of the absorbent article (20) within the vestibule (22) of a female wearer.

The absorbent article (20) of the present invention is desirably provided with sufficient capacity to absorb and retain the intended amount and type of bodily exudate(s). The absorbent capacity is provided by a fluid retentive core or absorbent generally identified as 46. For at least menstrual fluid, the absorbent article (20) may have an absorbent capacity ranging between no less than about 1 g/g up to no greater than about 30 g/g; although the approximate capacity of the absorbent article will vary according to, inter alia, the general design and intended disposition within the vestibule (22) of a female wearer. One of skill in the art will readily realize that the addition of superabsorbent material or coated superabsorbent material to the absorbent (46) typically has the effect of substantially increasing the absorbent capacity.

Describing the individual elements in greater detail, the absorbent (46) has an upper or body-facing surface and a lower surface (or surface opposed to the upper or body-facing surface) and may include any material capable of absorbing and/or adsorbing and thereafter retaining the intended bodily exudate(s). Suitable materials are also generally hydrophilic, compressible, and conformable. The absorbent (46) may be formed from any of the materials well known to those of ordinary skill in the art. Examples of such materials include, but are not limited to, various natural or synthetic fibers, multiple plies of creped cellulose wadding, fluffed cellulose fibers, rayon or other regenerated cellulose materials, wood pulp fibers or comminuted wood pulp fibers, airlaid material, textile fibers, a blend of polyester and polypropylene fibers, absorbent foams, absorbent sponges, superabsorbent polymers, coated superabsorbent polymers, fibrous bundles or nits, or any equivalent material or combination of materials. Also suitable for use would be hydrophobic material that has been rendered hydrophilic according to any of a number of suitable methods for so doing. The absorbent may also include degradable fibers. Other types of materials or structures may also be used, such as cellulose sponge or a sponge formed from elastomeric materials. When formed, the absorbent typically includes interstitial space or voids between the fibers or other materials. The total absorbent capacity of the absorbent (46) should, however, be compatible with the design exudate loading and the intended use of the absorbent article (20). Further, the size and absorbent capacity of the absorbent (46) may be varied. Therefore, the dimension, shape, and configuration of the absorbent (46) may be varied (e.g., the absorbent may have a varying thickness, or a hydrophilic gradient, or may contain superabsorbent material(s) and the like).

The absorbent (46) generally has a thickness, caliper or height (H), as illustrated at least in FIG. 3, measured along a line lying generally parallel to the z-axis. The thickness of the absorbent (46) may range from a minimum of no less than about 1 mm up to a maximum of no greater than about 10 mm; although the approximate thickness of the absorbent will vary according to, inter alia, the general design and intended disposition of the absorbent article (20) within the vestibule (22) of a female wearer.

The absorbent (46) also has a relatively low density that is deemed desirable for comfort. Generally, the density of the absorbent (46) may range up to about 0.5 g/cc; although the approximate density of the absorbent will vary according to, inter alia, the general design and intended disposition of the absorbent article (20) within the vestibule (22) of a female wearer.

The absorbent (46) may have a basis weight of about 600 gsm or less; although the approximate basis weight of the absorbent will vary according to, inter alia, the general design and intended disposition of the absorbent article (20) within the vestibule (22) of a female wearer. A specific example of a suitable absorbent would be similar to a coform material made of a blend of polypropylene and cellulose fibers and used in KOTEX® maxi pantiliners and obtainable from Kimberly-Clark Corporation, Neenah, Wis., USA.

The optional baffle (44) typically resides on the lower surface of the absorbent (46) and may be constructed from any desired material that is liquid-impermeable. Desirably, the baffle (44) will permit the passage of air and moisture vapor out of the absorbent (46), while blocking the passage of bodily fluid(s). An example of a suitable baffle material is a micro-embossed, polymeric film, such as polyethylene, polypropylene or polyester, having a minimum thickness of no less than about 0.025 mm and a maximum thickness of no greater than about 0.13 mm. Bicomponent films can also be used, as well as woven and nonwoven fabrics that have been treated to render them liquid-impermeable. An example of another suitable material is a closed-cell polyolefin foam. A closed-cell polyethylene foam may also work well.

The baffle (44) may be maintained in secured relation with the absorbent (46) by bonding all or a portion of the adjacent surfaces to one another. A variety of bonding methods known to one of skill in the art may be utilized to achieve any such secured relation. Examples of such methods include, but are not limited to, ultrasonics, thermal bonding, or the application of adhesives in a variety of patterns between the two adjoining surfaces. A specific example of a baffle material would be similar to a polyethylene film used on KOTEX® pantiliners and obtainable from Pliant Corporation, Schaumburg, Ill., USA.

The optional fluid permeable cover (42) has an upper surface and a lower surface, with the upper surface typically contacting the body of the wearer and receiving bodily exudate(s). The cover (42) desirably is made of a material that is flexible and non-irritating to the tissues within the vestibule (22) of a female wearer. As used herein, the term “flexible” is intended to refer to materials that are compliant and readily conform to the bodily surface(s), or that respond by easily deforming in the presence of external forces.

The cover (42) is provided for comfort and conformability and functions to direct bodily exudate(s) away from the body and toward the absorbent (46). The cover (42) should retain little or no liquid in its structure so that it provides a relatively comfortable and non-irritating surface next to the tissues within the vestibule (22) of a female wearer. The cover (42) can be constructed of any woven or nonwoven material that is easily penetrated by bodily fluids contacting its surface. Examples of suitable materials include rayon, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs and net material also work well. A specific example of a suitable cover material would be similar to a bonded carded web made of polypropylene and polyethylene used as a cover stock for KOTEX® pantiliners and obtainable from Sandler Corporation, Germany. Other examples of suitable materials are composite materials of a polymer and a nonwoven fabric material. The composite materials are typically in the form of integral sheets generally formed by the extrusion of a polymer onto a web of spunbond material. The fluid permeable cover (42) can also contain a plurality of apertures (not shown) formed therein which are intended to increase the rate at which bodily fluid(s) can penetrate into the absorbent (46).

A physiologically hydrous cover material is also suitable for use in the present invention. As used herein, the term “physiologically hydrous” is intended to connote a cover material that maintains a suitably moist interface between the tissues of the vestibule (22) and the absorbent article (20) when disposed in that vestibular environment; one that is benign respecting the requirements of comfort associated with the interposition of fabric or fabric-like structures within the moist tissue environment of the vestibule, keeping in mind as well the self-evident factor that the absorbent article is receiving bodily fluid(s) migrating through the vestibule and must conduct the same to the absorbent (46).

Thus, while not “hydrous” in the classic sense prior to use (inasmuch as the cover will be dry at that time) the cover (42) maintains (or at least does not interfere with the maintenance of) the proper moisture level or balance required within the vestibule (22).

The cover (42) can also have at least a portion of the surface treated with a surfactant to render the cover more hydrophilic. This results in permitting the insulting bodily fluid(s) to more readily penetrate the cover (42). The surfactant may also diminish the likelihood that the insulting bodily fluid(s), such as menstrual fluid, will flow off the cover (42) rather than being absorbed by the absorbent (46). One suitable approach provides for the surfactant to be substantially evenly distributed across at least a portion of the upper surface of the cover (42) that overlays the upper surface of the absorbent (46).

The cover (42) may be maintained in secured relation with the absorbent (46) by bonding all or a portion of the adjacent surfaces to one another. A variety of bonding methods known to one of skill in the art may be utilized to achieve any such secured relation. Examples of such methods include, but are not limited to, the application of adhesives in a variety of patterns between the two adjoining surfaces, entangling at least portions of the adjacent surface of the absorbent with the adjacent surface of the cover, or fusing at least portions of the adjacent surface of the cover to the adjacent surface of the absorbent.

The cover (42) typically resides on the upper surface of the absorbent (46), but alternatively can surround and partially or entirely enclose the absorbent. Alternatively, the cover (42) and the baffle (44) can have peripheries that extend outward beyond the periphery of the absorbent (46) and can be peripherally joined together to form an edge (64), as illustrated at least in FIG. 4. Utilizing known techniques, such as, for example, gluing, crimping, hot-sealing or the like, the edge (64) may be formed either entirely, so that the entire periphery of the absorbent (46) is circumscribed by their joinder, or the cover (42) and the baffle (44) can be partially peripherally joined. To minimize the possibility of irritation and/or discomfort to the wearer of the absorbent article (20), it is desired that the edge (64) and at least the area of the absorbent article immediately adjacent the edge be soft, compressible, and conformable. Any edge (64) so formed may have a width ranging from no less than about 0.5 mm up to no greater than about 10 mm; although the approximate width of any edge will vary according to, inter alia, the general design and intended disposition of the absorbent article (20) within the vestibule (22) of a female wearer. In other embodiments, the cover (42) and/or the baffle (44) can have a periphery that is coterminous with the periphery of the absorbent (46).

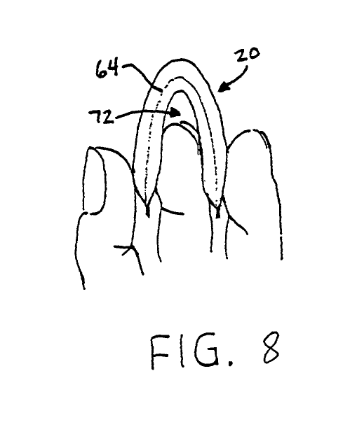

Positioned either on or substantially parallel to the principal longitudinal axis (L) of the absorbent (46), is, optionally, a desired axis of flexure (F). A desired axis of flexure (F) is generally positioned transversely, i.e., along the x direction, and may be off center from the principal longitudinal axis (L). Desirably, a desired axis of flexure (F) is aligned along the principal longitudinal axis (L). A desired axis of flexure (F) may result naturally from the dimensions, shape, and/or configuration of the absorbent (46), or the absorbent may be imparted with a weakened axis or region to create a desired axis of flexure. A desired axis of flexure (F) may also be formed by any of the techniques known to one of skill in the art, including, for example, scoring, pre-folding, slitting, embossing, or the like. Although a desired axis of flexure (F) is described herein as residing in the absorbent (46), one of skill in the art will readily appreciate that a desired axis of flexure may also be formed in either the cover (42), the baffle (44) and/or the absorbent; the cover and the baffle; the cover and the absorbent; or the baffle and the absorbent. Typically, the absorbent article (20) is folded along a desired axis of flexure (F), as illustrated at least in FIGS. 7 and 8, prior to disposition within the vestibule (22) of a female wearer.

The desired geometry of the absorbent article (20) (i.e., one in which the absorbent (46) has its maximum width (Wₘₐₓ) in one or both end regions) recognizes that a significant number of women do not have vaginal and urethral orifices located at the midpoint of a line extending longitudinally between the clitoris (40) and the posterior labial commissure (26). Although many drawings of the female anatomy illustrate the urethral orifice (38) near the anterior labial commissure (24) and the vaginal orifice (36) near the posterior labial commissure (26), with the vaginal orifice (36) being significantly larger than the urethral orifice (38), there is significant variation in the size and location of both orifices. The longitudinal distance between the urethral orifice (38) and the vaginal orifice (36) can vary significantly, as can the longitudinal distance between the clitoris (40) and the urethral orifice (38) and the longitudinal distance between the vaginal orifice (36) and the posterior labial commissure (26). In addition, the length of the labia may both vary significantly. For example, the longitudinal distance between the clitoris (40) and the urethral orifice (38) may range from about 0.5 to about 4 cm, while the longitudinal distance between the vaginal orifice (36) and the posterior labial commissure (26) may range from about 1 to about 5 cm. In addition to the variation in the previously described longitudinal distances, the longitudinal distance between the urethral (38) and vaginal (36) orifices can range from about 0.5 to about 4.5 cm. With such variations in distances, the absorbent article (20) of the present invention allows the wearer to position the end region having the maximum width of the absorbent (46) adjacent the desired orifice to intercept the intended bodily exudate(s). For example, if the intended bodily exudate is menstrual fluid and the vaginal orifice (36) is located closer to the posterior labial commissure (26), the wearer may position the end region having the maximum width (Wₘₐₓ) of the absorbent (46) under the vaginal orifice and thus closer to the posterior labial commissure. Alternatively, for example, if the intended bodily exudate is menstrual fluid and the vaginal orifice (36) is located closer to the clitoris (40), the wearer may position the end region having the maximum width (Wₘₐₓ) of the absorbent (46) under the vaginal orifice and thus closer to the clitoris. Alternatively still, for example, if the intended bodily exudate is menstrual fluid and the vaginal orifice (36) is located at the midpoint of a line extending longitudinally between the clitoris (40) and the posterior labial commissure (26), the wearer may select and position an absorbent article (20) having an appropriate geometry with the maximum width (Wₘₐₓ) of the absorbent (46) under the vaginal orifice with the region(s) having the minimum width (Wₘᵢₙ) of the absorbent oriented closer to either the clitoris or the posterior labial commissure, whichever is most comfortable for the female wearer. Consequently, the absorbent article (20) of the present invention may be reversibly disposed (i.e., with the minimum width (Wₘᵢₙ) of the absorbent (46) in a region closest to the clitoris (40), or with the minimum width (Wₘᵢₙ) of the absorbent in a region closest to the posterior labial commissure (26)) in the vestibule (22) of a female wearer. Such reversibility allows for a female wearer to maximize comfort and conformability by disposing the absorbent article (20) within her vestibule in an orientation which results in a customized fit best suited to the location of her principal urogenital members. The capability of affording a customized fit also allows individualized positioning or placement of the absorbent article (20) within the female wearer's vestibule (22). By allowing such individualized placement, the female wearer is able to dispose the absorbent article within her vestibule in an orientation where, in her opinion, (i) the most comfortable fit is obtained and (ii) she needs the maximum width (Wₘₐₓ) of the absorbent (46). Without desiring to be bound by theory, it is believed that the likelihood of leakage is minimized by affording a female wearer the opportunity to dispose the absorbent article (20) within her vestibule in an orientation that places the maximum width (Wₘₐₓ) of the absorbent (46) in close proximity to the chosen orifice to absorb and/or adsorb the desired exudate(s).

An absorbent article (20) with the desired geometry of the present invention, when folded along a desired axis of flexure (F) will have a profile in which the highest point along a desired axis of flexure (as measured in the z direction) is situated in the first end region (50) and/or the second end region (52), rather that in the central region (54). Even when not folded, however, the absorbent article (20) has a thickness, caliper or height (H), as illustrated at least in FIGS. 3 and 4, measured along a line laying generally parallel to the z-axis. The thickness of the absorbent article (20) may range from no less than about 1 mm up to no greater than about 10 mm; although the approximate thickness of the absorbent article will vary according to, inter alia, the general design and intended disposition of the absorbent article within the vestibule (22) of a female wearer.

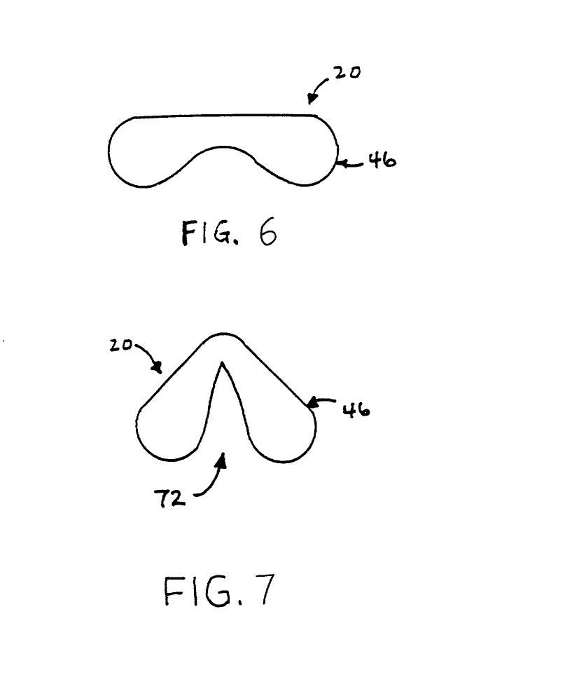

The absorbent article (20) is folded along a desired axis of flexure (F), as illustrated at least in FIGS. 7 and 8 prior to disposition within the vestibule (22) of the female wearer. When folded along a desired axis of flexure (F), the absorbent article (20) will form a recess (72) which protects the wearer's finger(s) from soiling when the absorbent article is disposed within the vestibule (22). Once inserted, the absorbent article (20) may have a tendency to unfold in an attempt to fill the vestibule and thus maintain the upper surface of the cover (42) in contact with the tissues of the vestibule (22). The absorbent article (20) may be resiliently biased along a desired axis of flexure (F) to increase the tendency of the absorbent article to unfold. Alternatively, the absorbent (46) of the absorbent article (20) may be thicker along its longitudinal edges as illustrated at least in FIGS. 6 and 7, thus also demonstrating a biasing effect, if desired, which is typically intended to allow the cover (42) to contact the tissues of the vestibule (22). An absorbent article (20) designed as described herein, however, does not necessarily require any additional features to maintain contact with the tissues of the vestibule (22) of the female wearer. The naturally moist surfaces of the tissues of the vestibule (22) typically demonstrate a tendency to maintain contact with the upper surface of the absorbent article (20).

In an alternate embodiment, the cover (42) can also have at least a portion of the surface treated with a suitable mucoadhesive to assist the absorbent article (20) in maintaining contact with the tissues of the vestibule (22) of the female wearer. These adhesives allow attachment of the absorbent article (20) to mucosal surfaces such as those of the inner labia. In use, the adhesive remains integrated with the absorbent article (20), which can still absorb menstrual fluid. Suitable mucoadhesives include copolymers of polyethylene-polypropylene-polyethylene (PEO-PPO-PEO) triblocks with chitosan and polyacrylic acid. Another representative example is the hydrophobically modified bioadhesive produced from hydroxyethyl methacrylate, methyl methacrylate, and acrylic acid. Yet another representative example is a polyacrylic acid based synthetic polymer known as Carbopol and described in J. Controlled Release 39 93, 1996. Further information regarding mucoadhesives may be found in “Physico-Chemical Properties of Water Insoluble Polymers Important to Mucin/Epithelial Adhesion,” H. Park and J. Robinson, J. Controlled Release, Vol. 2, (1985), pp. 47-57; and in “Development and Evaluation of a Mucoadhesive Drug Delivery System for Dual-Controlled Delivery of Nonoxynol-9,” C. Lee and Y. Chien, J. Controlled Release, Vol. 39 (1996), pp. 91-103, both of which are incorporated herein by reference. Any suitable mucoadhesive familiar to one skilled in the art can be used.

As noted above, the wearer may fold the absorbent article (20) along a desired axis of flexure (F) prior to disposition within the vestibule (22). The wearer may, therefore, hold the folded absorbent article (20) at the longitudinal sides (58, 60) and begin disposition as illustrated at least in FIG. 8. The absorbent article (20) may then be disposed within the vestibule (22) by the wearer exerting a force with a finger or fingers positioned in the recess (72) formed by the folded absorbent article.

A therapeutic agent delivery system including a therapeutic agent can be produced integrally with the absorbent article (20). For the purposes of this invention, any therapeutic agent that will be delivered across the non-cornified epithelium of the labia for the purposes of treating diseases or conditions such as, for example, dysmenorrhea, can be used. Alternatively, or in addition, therapeutic and other beneficial agents such as vitamins, hormones, moisturizers, antifungal agents, antibacterial agents, pro-biotic agents that promote the growth of normal vaginal bacterial flora, and the like may be similarly delivered.

Therapeutic agents for use in the invention are absorbable through the non-cornified epithelium of the labia and travel to the uterus by a unique portal of veins and arteries that are known to exist between the vagina, the cervix, and the uterus. This anastomosis eliminates so-called first pass metabolism by the liver, effectively delivering higher concentrations of therapeutic agent to the uterus than would otherwise be available via oral dosing. One skilled in the art knows the efficacy of therapeutic agents in such an application when introduced at a particular anatomical location. For example, when the therapeutic agent is selected to treat dysmenorrhea, it preferably is selected from the group consisting of nonsteroidal anti-inflammatory drugs (NSAIDs), prostaglandin inhibitors, COX-2 inhibitors, local anesthetics, calcium channel blockers, potassium channel blockers, β-adrenergic agonists, leukotriene blocking agents, smooth muscle inhibitors, and drugs capable of inhibiting dyskinetic muscle contraction.

COX-2 inhibitors, such as Celecoxib, Meloxicam, Rofecoxib, and Flosulide are novel anti-inflammatory and analgesic compounds. These compounds effectively inhibit production of COX-2 (cyclooxygenase-2) enzyme that is induced by pro-inflammatory stimuli in migratory cells and inflamed tissue. Because COX-2 is also involved in reproductive processes, selective COX-2 inhibitors will reduce uterine contractions in pre-term labor and relieve painful uterine contractions associated with dysmenorrhea by blocking prostaglandin receptors in the uterus. Additionally, they may reduce endometrial bleeding.

Preferred NSAIDs include Aspirin, Ibuprofen, Indomethacin, Phenylbutazone, Bromfenac, Sulindac, Nabumetone, Ketorolac, Mefenamic Acid, and Naproxen. Preferred local anesthetics include Lidocaine, Mepivacaine, Etidocaine, Bupivacaine, 2-Chloroprocaine hydrochloride, Procaine, and Tetracaine hydrochloride. Preferred calcium channel antagonists include Diltaizem, Israpidine, Nimodipine, Felodipine, Verapamil, Nifedipine, Nicardipine, and Bepridil. Preferred potassium channel blockers include Dofetilide, E-4031, Imokalant, Sematilide, Ambasilide, Azimilide, Tedisamil, RP58866, Sotalol, Piroxicam, and Ibutilide. Preferred β-adrenergic agonists include Terbutaline, Salbutamol, Metaproterenol, and Ritodrine. Vasodilators, which are believed to relieve muscle spasm in the uterine muscle, include nitroglycerin, isosorbide dinitrate, and isosorbide mononitrate.

Examples of beneficial botanicals may include, but are not limited to, Agnus castus, aloe vera, comfrey, calendula, dong quai, black cohosh, chamomile, evening primrose, Hypericum perforatum, licorice root, black currant seed oil, St. John's wort, tea extracts, lemon balm, capsicum, rosemary, Areca catechu, mung bean, borage seed oil, witch hazel, fenugreek, lavender, and soy. Vaccinium extracts commonly derived from many members of the heath family, cranberries such as blueberries, and azaleas (Rhododendron spp.) as well as from red onion skin and short and long red bell peppers, Beta vulgaris (beet) root extract, and capsanthin may also be used. Other berries that have applicability are whortleberry, lingenberry, chokeberry, sweet rowan, rowanberry, seabuckhrouberry, crowberry, strawberries, and gooseberries.

These botanicals can be combined with other beneficial agents including, but are not limited to, vitamins, calcium, magnesium, hormones, analgesics, prostaglandin inhibitors, prostaglandin synthetase inhibitors, leukotriene receptor antagonists, essential fatty acids, sterols, anti-inflammatory agents, vasodilators, chemotherapeutic agents, and agents to treat infertility.

These beneficial therapeutic agents promote epithelial health in the vaginal region by delivering botanical ingredients with a feminine care device. The idea is to modulate the vaginal environment to enhance the wellness of this anatomical region. These benefits can be rather simple, for example increasing comfort by providing moisturization and/or lubricity. These benefits can also be more complex, for example modulating epithelial cell function to address vaginal atrophy. The beneficial therapeutic agents may reduce negative sensations such as stinging, burning, itching, etc, or introduce positive sensations to improve comfort.

For example, many therapeutic benefits have been ascribed to a large number of different botanical preparations. Preparations may include water-in-oil emulsions, oil-in-water emulsions, gel, liquid, dispersion, powder, and anhydrous systems, ointment, or salve, such as a botanical oil in an anhydrous base (e.g., petrolatum), or polyethylene glycol based systems. Also, botanicals are often prepared or extracted under conditions to generate water-soluble or oil-soluble extracts. These extracts are usually compositionally different and may have different skin and vaginal health benefits. Processing conditions will have an effect on the type of formulation that can be used and this will restrict the type of botanical (water or oil type) selected. Therefore, wide ranges of botanicals have utility in this invention. Botanicals can possess a variety of actives and activities that can include, but are not necessarily limited to, analgesics, antimicrobials, pro-biotic agents, anti-inflammatory compounds, anti-virals, enzymes, enzyme inhibitors, enzyme substrates, enzyme cofactors, ions, ion chelators, lipids, lipid analogs, lipid precursors, hormones, inflammatory mediators, inflammatory agonists, oxidants, antioxidants, humectants, growth factors, sugars, oligosaccarides, polysaccarides, vasodilators, and potential combinations thereof. It is understood that, for the purposes of this invention, the botanicals can be combined with any number of non-botanical active ingredients as well.

As a specific example, the NSAID mefenamic acid is commonly prescribed to treat dysmenorrhea. In this example, the cover (42) of the absorbent article (20) is modified with a mixture of a mucoadhesive and mefenamic acid. The mixture is applied to an application region that may encompass all or a portion of the absorbent article (20). As described above, the mucoadhesive helps hold the pad in place during use. The NSAID is delivered across the non-cornified epithelium of the labia where it becomes systemic to relieve symptoms of dysmenorrhea such as cramping and pain. The absence of a stratum corneum on the inside of the labia facilitates more rapid transport of NSAID into the tissue relative to the rate of delivery across skin containing a keratinized stratum corneum. In use, the adhesive remains integrated with the absorbent article (20), which can still absorb menstrual fluid, but allows for diffusion of the NSAID out of the adhesive and into the vaginal tissue.

The therapeutic agent delivery system may also include carrier components to promote the functionality of the therapeutic agent. For example, the carrier components may assist the therapeutic agent in absorbing into, or adhering onto, the absorbent article (20). The carrier components may assist the release of the therapeutic agent from the absorbent article (20), or assist in the absorbency of the therapeutic agent into the labial epithelium. The use of excipients to facilitate the formulation, delivery, stability, and aesthetic properties of a drug delivery system is well known to those familiar with the art.

In one embodiment, the therapeutic agent and the therapeutic agent delivery system are applied to the outer surface of the absorbent article (20), predominantly to the surfaces that will be in contact with the labial epithelium. In an alternate embodiment, the formulation including a therapeutic agent may be applied to degradable fibers in or on the absorbent article (20). In another embodiment, the formulation including a therapeutic agent may be interspersed through the interstitial space in the absorbent.

As an example, the catamenial absorbent article (20) includes a porous cover (42) that contains a therapeutic agent. Typically, such an absorbent article (20) would have a cover (42) formed from spunbond fibers of a hydrophobic polymeric material, e.g., a spunbond polypropylene cover layer, with a therapeutic agent coated on the outside of the fibers.

It may not be necessary to impregnate the entire absorbent body of an absorbent product, such as an absorbent article (20), with the therapeutic agent. Optimum results, both economically and functionally, can often be obtained by concentrating the material on or near an outer surface where it will be most effective during use. The therapeutic agent may also be applied, however, in other locations around and within an absorbent article to control the release of the therapeutic agent, or if those other locations are more advantageous for a given agent or a given condition.

The formulation including a therapeutic agent may be applied to the absorbent article (20) using conventional methods for applying a formulation including a therapeutic agent to the desired absorbent article (20). For example, a unitary absorbent article (20) may be dipped directly into a bath having the agent and then can be dried. The formulation including a therapeutic agent when incorporated on and/or into the absorbent article materials may be fugitive, loosely adhered, bound, or any combination thereof. As used herein the term “fugitive” means that the formulation including a therapeutic agent is capable of migrating through the absorbent article materials. For example, a therapeutic agent may be blended together with a polymeric material that is to be processed into a component of an absorbent or non-absorbent product.

Alternatively, a formulation including a therapeutic agent may be applied directly onto an individual layer of material before it is incorporated into an article to be manufactured, such as an absorbent product. For example, an aqueous solution containing a therapeutic agent can be applied by any method known in the art onto the surface of a porous cover sheet or absorbent layer designed to be incorporated into an absorbent product. This can be done either during the production of the individual layer or during a fabrication process that incorporates the layer into the article being manufactured.

Nonwoven webs coated with a formulation including a therapeutic agent can be prepared by conventional processes. For example, a formulation including a therapeutic agent can be applied to one or both sides of a traveling web. Those skilled in the art will appreciate that the application can be carried out as an inline treatment or as a separate, offline treatment step. A web, such as a spunbond or meltblown nonwoven, can be directed over support rolls to a treating station including rotary spray heads for application to one side of web. An optional treating station may include rotary spray heads to apply a formulation including a therapeutic agent to the opposite side of the web. Each treatment station generally receives a supply of treating liquid from a reservoir. The treated web may then be dried if needed by passing over dryer cans or other drying means and then wound as a roll or converted to the use for which it is intended. Alternative drying apparatus such as ovens, through air dryers, infra red dryers, air blowers, and the like may also be utilized. Another method of application would utilize the drug containing mucoadhesive formulation as a hot melt composition. Contact transfer or hot melt spray application processes could be used to treat the absorbent product with the therapeutic formulation.

Active ingredients, such as pharmaceutical compounds (e.g., histidines, anti-inflammatory drugs, calcium or potassium channel blockers), antimicrobials, anesthetics, hormones or hormone inhibitors, pH control agents, and the like, can be provided in any known drug delivery medium that is placed within the absorbent article (20). An example is microencapsulation of the active ingredient in starch, dextran, or other degradable or soluble materials, such that microcapsules placed in the absorbent material of the tampon can permit gradual release of the active ingredient upon wetting, an increase in temperature, or physical contact. Another type of delivery system is the use of polymeric transport systems, which are materials that absorb materials and will release these materials when applied to a substrate.

Combining the active ingredient with a hydrophobic material such as a solidifying agent; wax, solid ester, solid fatty alcohol or acid, hydrogenated vegetable oil, solid triglycerides, natural soft solid materials (i.e., cocoa butter), solid alkyl silicones, and the like, allows gradual diffusion of the active ingredient from the hydrophobic material to the body of the wearer, while preventing loss of the active ingredient during gushing of body fluids. In one embodiment, the solidifying agent can be solid at room temperature but can soften at body temperature to increase the release rate of the active ingredient once the product has been in contact with the body for a period of time.

The active ingredient may be combined with a hydrogel or superabsorbent material. Upon wetting, the hydrogel or superabsorbent material swells, resulting in increased delivery of the active ingredient from the swollen material.

The active ingredient may also be combined with a substantially hydrophobic emollient or lotion that can resist being washed away by aqueous body fluids but which can nevertheless transfer to body surfaces during use to enhance drug delivery.

Another example is the use of polyethylene glycols with molecular weights greater than 720 as solidifying agents. The active ingredient can be solubilized or dispersed in polyethylene glycols, which are water dispersible materials. Contact with water containing body fluids will slowly dissolve the polyethylene glycol and release the active ingredient to the body surface.

Finally, the active ingredient may be placed within a pouch in the absorbent article (20), which can release active ingredients by diffusion through a permeable membrane, rupture or degradation of a portion of the wall of the pouch, or active deployment wherein, for example, a material in the pouch or reservoir swells upon wetting and forces expulsion of the active ingredient, or a foam is generated to carry the active ingredient out of the pouch.

Nonwoven or film components such as a liquid-pervious cover layer or other component can also be combined with active ingredients in a variety of means. The active ingredient can be attached to the surface of the nonwoven or film, or may be incorporated into the solid matrix. For example, an active ingredient can be blended in one or more polymer phases prior to manufacture of the nonwoven or film, or can be added into the solid phase as a post treatment by a variety of means, including delivery in a supercritical fluid carrier. With polyolefin polymers and other compounds, the presence of supercritical carbon dioxide, for example, causes substantial swelling of the polymer, creating large pore spaces in the swollen state into which an active ingredient can diffuse. Removal of the supercritical carbon dioxide then causes reversal of the swelling, resulting in trapping of the active ingredient within the solid matrix of the nonwoven or film, with the possibility for gradual release of the active ingredient from the matrix when in contact with biological membranes or fluids, especially upon wetting.

Alternately, vehicles with various degrees of complexity can be used ranging from simple vehicles made of a singular substance to gels, liquids, emulsions, solids, powders or to even more complex vehicles such as those containing liposomes or particulate materials bearing specific ligands with which to target the agent to particular locations within the vaginal environment. In other embodiments, the device could include degradable hollow fibers or other structures wherein the cavity is filled with the agent. In this way the material would be released only in response to specific events. In still other embodiments, the absorption of the therapeutic agent can be augmented with penetration enhancers.

Apertured webs can also be used to contain an active ingredient, either as a substrate or component in a laminated structure. The webs that can be used include those of Tredegar Corp. and AET Specialty Nets & Nonwovens, including the latter's includes DELNET-brand geometric apertured fabrics, DELNET-EP-brand coextruded adhesive fabrics, PLASTINET-brand biplanar netting and sleeving, STRATEX-brand engineered laminated structures, and DELPORE-brand, DELGUARD-brand and DELSORB-brand meltblown nonwoven fabrics, any of which can be treated with or combined with active ingredients. Active ingredients can also be provided as an internal component of a laminated structure, such as a central layer in a laminate between two film layers.

Foam components can also be combined with active ingredients. Active ingredients can be directly mixed with the solid matter forming the matrix of the foam, or can be contained as a solid phase such as particulates or as a viscous phase within the open or enclosed cells of the foam. Release of the active ingredient can occur upon wetting, wither by solvating the active ingredient from the solid matrix, dissolving the walls of an encapsulating medium, or permitting a diffusion pathway back to mucosal membranes. Foam matrices can include superabsorbent material; regenerated cellulose; synthetic polymers such as polyurethane; gelatin or other protein-based compositions such as those derived from albumin; High-lnternal-Phase-Ratio Emulsions (HIPE) technology such as that disclosed in U.S. Pat. No. 5,652,194, “Process for Making Thin-Wet Absorbent Foam Materials for Aqueous Body Fluids,” issued Jul. 29, 1997 to Dyer et al.; and fiber-based foam compositions such as those disclosed in U.S. Pat. No. 6,261,679, “Fibrous Absorbent Material and Methods of Making the Same,” issued Jul. 17, 2001 to F-J. Chen et al.

Cellulose fibers can be combined with active ingredients in a variety of ways, including attachment by chemical or physiochemical means such as van der Waals forces, covalent bonds or ionic bonds; physical entanglement (being mechanically trapped by the porous structure); or lumen loading, wherein the active ingredient is chemically or mechanically deposited into the hollow lumen or core of a natural cellulose fiber or a synthetic fiber, as disclosed in U.S. Pat. No. 4,510,020, issued to H. V. Green et al., Apr. 9, 1985; or U.S. Pat. No. 5,096,539, issued to G. G. Allan, Mar. 17, 1992. The same can be done for hollow non-cellulose fibers. Cellulose webs can also be impregnated or coated with active ingredients, either alone or in combination with hydrophobic matter, hydrogels, or other carriers, as disclosed, for example, in U.S. Pat. No. 5,990,377, “Dual-zoned Absorbent Webs,” issued Nov. 23, 1999 to F-J. Chen et al.

Active ingredients can also be combined with an active deployment means that physically moves the active ingredient after being triggered by wetting or an increase in temperature. For example, the active deployment means can comprise generation of foam or bubbles in an effervescent effect that can move the active ingredient from within the absorbent article (20) toward the body of the user, triggered by contact with an aqueous fluid, for example. A swellable material placed with the active ingredient in a pouch with a liquid-pervious inelastic wall can swell upon wetting and force expulsion of the active ingredient from the pouch.

In an alternate embodiment, a reservoir (76) (see FIG. 4) within the absorbent article (20) is provided in which to locate the therapeutic agent. The agent may be stored within the reservoir in various forms and in varying dosages. For example, the agent may be placed in the reservoir in liquid form, in solid form, in semi-solid form, or in an encapsulated form. The agent may be formulated to act immediately upon use of the absorbent article (20), or in a time-release manner. The agent may be activated by pressure from application, or from pressure, heat, or humidity in the labial environment. The agent may be placed by the manufacturer of the absorbent article (20) or the absorbent article user as needed.

The therapeutic agent may be combined into a formulation that may contain other additives or carrier components as appropriate for the desired result so long as the additives or carrier components do not have a major detrimental effect on the activity of the therapeutic agent. Examples of such additives include additional conventional surfactants, such as esters like myreth-3-myristate, ethoxylated hydrocarbons, or ionic surfactants, or co-wetting aids such as low molecular weight alcohols. The formulation is desirably applied from high solids, advantageously 80% or less solvent or water, so as to minimize drying and its attendant costs and deleterious effects. The treating formulation including a therapeutic agent may be applied in varying amounts depending on the desired results and application. Those skilled in the art can readily select the actual amount based on the teaching of this application. For example, a catamenial absorbent article (20) designed to be in intimate contact with the labial epithelium might require substantially less therapeutic agent than an agent taken orally due to the absence of first pass liver metabolism as previously discussed.

It will be recognized by those skilled in this art that a therapeutic agent may be used as an internal additive, that is, added to the polymer melt directly or in a concentrate form. After fiber formation, such additives can migrate to the fiber surface and impart the desired effect. For further discussion of internal addition of additives, see for example, U.S. Pat. No. 5,540,979, the contents of which are incorporated herein by reference. The substrate basis weight is not critical and may vary widely depending on the application. The thermal and oxidation stability of the therapeutic agent must be compatible with the temperature and rheology required for melt processing.

The formulation including a therapeutic agent of the present invention can be prepared and applied in other suitable forms, including without limitation, aqueous solutions, emulsions, lotions, balms, gels, salves, powders, ointments, muco-adhesives, boluses, suppositories, and the like. The formulations of this invention may also contain preservatives. Compounds that can impart greater viscosity, such as polyethylene glycol and the like, may also be added to the formulations of this invention. Generally, higher viscosity formulations are preferred to create formulations that will tend to remain in the vagina for a relatively long time period after administration.

A therapeutic agent formulation may additionally employ one or more conventional pharmaceutically-acceptable and compatible carrier materials useful for the desired application. The carrier can be capable of co-dissolving or suspending the materials used in the formulation. Carrier materials suitable for use in the instant formulation including a therapeutic agent, therefore, include those well-known for use in the pharmaceutical, cosmetic, and medical arts as a basis for ointments, lotions, creams, salves, aerosols, suppositories, gels, powders, and the like.

As various changes could be made in the foregoing absorbent articles without departing from the scope of the invention, it is intended that all matter contained in the above description and shown in the accompanying drawings shall be interpreted as illustrative and not in a limiting sense. Accordingly, this invention is intended to embrace all such alternatives, modifications and variations that fall within the spirit and scope of the appended claims.

We claim:

1. An absorbent device configured for partial disposition within the vestibule of a wearer, and adapted to deliver a therapeutic agent, the device comprising:
a fluid-absorbent body having an application region for projection within the vestibule; and
a formulation including a therapeutic agent positioned substantially within the application region.

2. The device of claim 1, wherein the body includes a cover, and wherein the therapeutic agent is formed with the cover.

3. The device of claim 1, wherein the body includes a cover having a surface, and wherein the therapeutic agent is coupled to the surface.

4. The device of claim 1, wherein the body includes an absorbent, and wherein the formulation including the therapeutic agent is formed with the absorbent.

5. The device of claim 1, wherein the body includes an absorbent, and wherein the formulation including the therapeutic agent is contained in the absorbent.

6. The device of claim 1, wherein the body includes an absorbent having a surface, and wherein the therapeutic agent is coupled to the surface.

7. The device of claim 1, further comprising a reservoir within the application region, wherein the therapeutic agent is located substantially within the reservoir.

8. The device of claim 7, wherein the application region has a surface, and wherein the reservoir is in communication with the surface.

9. The device of claim 7, wherein the application region has a surface, and wherein the reservoir is located under the surface.

10. The device of claim 1, wherein the formulation including the therapeutic agent is substantially a liquid.

11. The device of claim 1, wherein the therapeutic agent is an emulsion.

12. The device of claim 1, wherein the therapeutic agent is a powder.

13. The device of claim 1, wherein the therapeutic agent is a gel.

14. The device of claim 1, wherein the therapeutic agent is an ointment.

15. The device of claim 1, wherein the therapeutic agent is a salve.

16. The device of claim 1, wherein the formulation including the therapeutic agent is substantially a solid.

17. The device of claim 1, wherein the formulation including the therapeutic agent is substantially a semi-solid.

18. The device of claim 1, wherein the formulation including the therapeutic agent is encapsulated.

19. The device of claim 1, wherein pressure applied by the user to the fluid-absorbent body releases the formulation including the therapeutic agent from the application region.

20. The device of claim 1, wherein pressure applied by the user to the fluid-absorbent body releases the formulation including the therapeutic agent to the application region.

21. The device of claim 1, wherein the therapeutic agent is adapted to treat dysmenorrhea.

22. The device of claim 1, wherein the therapeutic agent is selected from the group consisting of aspirin, ibuprofen, indomethacin, phenylbutazone, bromfenac, sulindac, nabumetone, ketorolac, mefenamic acid, and naproxen.

23. The device of claim 1, wherein the therapeutic agent is selected from the group consisting of Lidocaine, Mepivacaine, Etidocaine, Bupivacaine, 2-Chloroprocaine hydrochloride, Procaine, and Tetracaine hydrochloride.

24. The device of claim 1, wherein the therapeutic agent is selected from the group consisting of Diltaizem, Israpidine, Nimodipine, Felodipine, Verapamil, Nifedipine, Nicardipine, and Bepridil.

25. The device of claim 1, wherein the therapeutic agent is selected from the group consisting of Dofetilide, E-4031, Imokalant, Sematilide, Ambasilide, Azimilide, Ted isamil, RP58866, Sotalol, Piroxicam, and Ibutilide.

26. The device of claim 1, wherein the therapeutic agent is selected from the group consisting of Terbutaline, Salbutamol, Metaproterenol, and Ritodrine.

27. The device of claim 1, wherein the therapeutic agent is selected from the group consisting of nitroglycerin, isosorbide dinitrate, and isosorbide mononitrate.

28. The device of claim 1, wherein the therapeutic agent is selected from the group consisting of Celecoxib, Meloxicam, Rofecoxib, and Flosulide.

29. The device of claim 1, wherein the therapeutic agent is selected from the group consisting of: Agnus castus, aloe vera, comfrey, calendula, dong quai, black cohosh, chamomile, evening primrose, Hypericum perforatum, licorice root, black currant seed oil, St. John's wort, tea extracts, lemon balm, capsicum, rosemary, Areca catechu, mung bean, borage seed oil, witch hazel, fenugreek, lavender, and soy.

30. The device of claim 1, wherein the therapeutic agent is a Vaccinium extract derived from a plant selected from the group consisting of: heath, cranberries, blueberries, azaleas, red onion skin, short red bell peppers, long red bell peppers, beet root extract, and capsanthin.

31. The device of claim 1, wherein the therapeutic agent is selected from the group consisting of: whortleberry, lingenberry, chokeberry, sweet rowan, rowanberry, seabuckhrouberry, crowberry, strawberries, and gooseberries.

32. The device of claim 1, wherein the therapeutic agent is a combination of a botanical and a beneficial agent selected from the group consisting of: vitamins, calcium, magnesium, hormones, analgesics, prostaglandin inhibitors, prostaglandin synthetase inhibitors, leukotriene receptor antagonists, essential fatty acids, sterols, anti-inflammatory agents, vasodilators, chemotherapeutic agents, and agents to treat infertility.

33. The device of claim 1, wherein the formulation includes a ligand adapted to target the therapeutic agent.

34. The device of claim 1, wherein the body includes a surface, and wherein the formulation including a therapeutic agent is applied to the surface.

35. The device of claim 1, wherein the body is constructed from a material, and wherein the formulation including a therapeutic agent is applied to the material before the body is constructed.

36. The device of claim 1, wherein the body includes an apertured web, and wherein the formulation including a therapeutic agent is contained in the apertured web.

37. The device of claim 1, wherein the formulation including a therapeutic agent is applied to degradable fibers.

38. The device of claim 1, wherein the body has an interstitial space, and wherein the formulation including a therapeutic agent is interspersed within the interstitial space.

39. The device of claim 1, wherein the formulation including a therapeutic agent includes a hydrogel material.

40. The device of claim 1, wherein the formulation including a therapeutic agent includes a foam component.

41. The device of claim 1, wherein the formulation including a therapeutic agent includes a polymeric material.

42. A method for producing an absorbent device configured for partial disposition within the vestibule of a wearer, and adapted to deliver a therapeutic agent, the method comprising:
manufacturing an absorbent device having a fluid-absorbent body having an application region for projection within the vestibule; and
locating a formulation including the therapeutic agent substantially within the application region.

43. The method of claim 42, further comprising applying a mucoadhesive adapted to enhance the contact between the absorbent article and a non-cornified epithelium of the wearer.

44. The method of claim 42, wherein the manufacturing act includes manufacturing the body with a cover, wherein the formulation including the therapeutic agent is formed with the cover.

45. The method of claim 42, wherein the manufacturing act includes manufacturing a body with cover having a surface, wherein the formulation including the therapeutic agent is coupled to the surface.

46. The method of claim 42, wherein the manufacturing act includes manufacturing the method such that pressure applied by the wearer to the fluid-absorbent body releases the formulation including the therapeutic agent from the application region.

47. The method of claim 42, wherein the body has a surface, and wherein the locating act includes applying the formulation including a therapeutic agent to the surface.

48. The method of claim 42, wherein the manufacturing act includes manufacturing the body from a material, and wherein the locating act includes applying the formulation including a therapeutic agent to the material before the body is manufactured.

49. The method of claim 42, wherein the manufacturing act includes manufacturing the body to include an apertured web, and wherein the locating act includes containing the formulation including a therapeutic agent in the apertured web.

50. The method of claim 42, wherein the locating act includes producing the formulation including a therapeutic agent integrally with the device.

51. An absorbent device configured for partial disposition within the vestibule of a wearer, and adapted to deliver a therapeutic agent, the device comprising:
a fluid-absorbent body having an application region for projection within the vestibule; and
a means for carrying a formulation including the therapeutic agent within the application region.

52. The device of claim 51, wherein the application region has a surface, and wherein the carrying means is substantially positioned adjacent the surface.

53. The device of claim 51, wherein the application region has a reservoir, and wherein the carrying means is substantially positioned within the reservoir.

54. A method of producing an absorbent article configured for partial disposition within the vestibule of a wearer and adapted to deliver a therapeutic agent, the method comprising:
treating a portion of a porous nonwoven sheet formed from hydrophobic polymer with a formulation including the therapeutic agent; and
forming the absorbent article so as to include absorbent material, such that the absorbent article has an application region for projection within the vestibule, and such that the portion of the porous nonwoven sheet at least partially covers the application region of the absorbent article.

55. A method of delivering a therapeutic agent through the non-cornified epithelium of the labia of a wearer, the method comprising:
disposing an absorbent article at least partially within the vestibule of the wearer, the device being adapted to contact the non-cornified epithelium and deliver the therapeutic agent.

56. The method of claim 55, wherein pressure applied by the wearer to the absorbent article releases the therapeutic agent.

57. The method of claim 55, wherein the pressure is applied by the wearer during placement of the absorbent article.

58. The method of claim 55, wherein the pressure is applied by the wearer during use of the absorbent article.

59. The method of claim 55, wherein delivery of the therapeutic agent is effected by melting a solid.

60. The method of claim 55, wherein delivery of the therapeutic agent is effected by rupturing a capsule.

61. The method of claim 55, wherein delivery of the therapeutic agent is effected by melting a semi-solid.

62. The method of claim 55, wherein delivery of the therapeutic agent is effected by combining the therapeutic agent with a mucoadhesive that enhances the contact between the absorbent article and the non-cornified epithelium.

63. An absorbent device comprising:
a fluid-absorbent body having an application region for projection within the vestibule of a wearer; and
a formulation including a therapeutic agent, wherein the application region is adapted to contact and deliver the therapeutic agent through the non-cornified epithelium of the labia.
